(19) United States
(12) Patent Application Publication
(10) Pub. No.: US 2012/0178979 A1
(43) Pub. Date: Jul. 12, 2012

(54) METHOD FOR PREPARING A MULTI-METAL CATALYST HAVING AN OPTIMIZED SITE PROXIMITY

(75) Inventors: Priscilla Avenier, Grenoble (FR); Sylvie Lacombe, Vernaison (FR); Herve Cauffriez, Charly (FR)

(73) Assignee: IFP Energies nouvelles, RUEIL-MALMAISON CEDEX (FR)

(21) Appl. No.: 13/381,281

(22) PCT Filed: Jun. 15, 2010
(86) PCT No.: PCT/FR2010/000443

§ 371 Date: Mar. 30, 2012

(30) Foreign Application Priority Data
Jul. 1, 2009 (FR) 09/03.226

(51) Int. Cl. C07C 15/02; B01J 27/186; B01J 37/08; B01J 27/185

(52) U.S. Cl. 585/400; 502/213

(57) ABSTRACT

The invention concerns a process for preparing a catalyst comprising at least one metal M from the platinum group, tin, a phosphorus promoter, a halogenated compound, a porous support and at least one promoter X1 selected from the group constituted by gallium, indium, thallium, arsenic, antimony and bismuth. The promoter or promoters X1 and the phosphorus are introduced during one or more sub-steps a1) or a2), the sub-step a1) corresponding to synthesis of the precursor of the main oxide and sub-step a2) corresponding to shaping the support. The tin is introduced during at least one of sub-steps a1) and a2). The product is dried and calcined before depositing at least one metal M from the platinum group. The ensemble is then dried in a stream of neutral gas or a stream of gas containing oxygen, and then is dried. The invention also concerns the use of a catalyst obtained by said process in catalytic reforming or aromatics production reactions.

### FIELD OF THE INVENTION

The present invention relates to the field of hydrocarbon conversion, and more specifically to reforming hydrocarbon feeds in the presence of a catalyst to produce gasoline cuts. The invention also relates to improved catalytic formulations based on at least one metal from the platinum group for use in said conversion, as well as to their mode of preparation.

### PRIOR ART

Many patents describe adding promoters to platinum-based catalysts in order to improve their performance as regards hydrocarbon feed reforming. Thus, patent U.S. Pat. No. 2,814,599 describes adding promoters such as gallium, indium, scandium, yttrium, lanthanum, thallium or actinium to catalysts based on platinum or palladium.

Patent U.S. Pat. No. 4,522,935 describes reforming catalysts comprising platinum, tin, indium and a halogenated compound deposited on a support in which the indium/platinum atomic ratio is more than 1.14.

Patent FR 2 840 548 describes a catalyst in the form of a homogeneous bed of particles comprising an amorphous matrix, at least one noble metal, at least one halogen and at least one additional metal. Said additional metal is preferably selected from the group constituted by tin, germanium, lead, gallium, indium, thallium, rhenium, manganese, chromium, molybdenum and tungsten.

Phosphorus is also known to increase the yields of hydrocarbon compounds containing strictly more than 4 carbon atoms (C5+), in particular aromatic products. That property is claimed in patents U.S. Pat. No. 2,890,167, U.S. Pat. No. 3,706,815, U.S. Pat. No. 4,367,137, U.S. Pat. No. 4,416,804, U.S. Pat. No. 4,426,279 and U.S. Pat. No. 4,463,104. More recently, patent US 2007/0215523 described that adding diluted quantities of phosphorus, less than 1% by weight, stabilizes the support by allowing better retention of specific surface area and chlorine during its use in catalytic reforming processes.

The patents U.S. Pat. No. 6,864,212 and U.S. Pat. No. 6,667,270 describe a support containing bismuth and phosphorus distributed in a homogeneous manner and used for preparing a catalyst for the catalytic reforming of hydrotreated naphtha. According to those patents, adding bismuth alone to the support can slow down the formation of coke and the decline in activity, but the C5+ yield is reduced at the same time, while adding phosphorus alone increases that yield without improving the stability of the catalyst. The combination of those two elements can further slow coke formation down while at the same time having better selectivities for Bi contents in the range 0.10% to 0.06% by weight, and a P content of 0.3% by weight. Those two patents do not claim other elements.

Solid state NMR spectroscopy, in particular magic angle spinning (MAS) ³¹P NMR, has been used intensively for the characterization of the environment of phosphorus atoms in aluminophosphate type materials. Materials of that type have a chemical shift range of 0 to −30 ppm, as described in the articles by Sayari et al (Chem Mater 8, 1996, 2080-2088) or by Blackwell et al (J Phys Chem 92, 1988, 3965-3970; J Phys Chem 88, 1984, 6135-6139). In that range of shifts, fully condensed sites for the phosphorus in the alumina can be distinguished from sites with incomplete condensation, as indicated by Huang et al (J Am Chem Soc, 127(8), 2005, 2731-2740). However, in order to determine the nature of the phosphorus environment more precisely, that single series of experiments is not sufficient. Coupling this series, as ¹→³¹P cross polarization magic angle spinning (CP MAS) NMR, can, for example, distinguish protonated environments of the phosphorus and thus discriminate surface atoms from atoms in the matrix.

Further, monitoring the adsorption of carbon monoxide onto supported metallic catalysts using infrared spectroscopy is a means of acquiring information regarding the electron density of metallic particles or the acidity of the support, depending on whether the adsorption occurs at ambient temperature or at that of liquid nitrogen. In the case, for example, of a platinum-based catalyst supported on alumina, at ambient temperature, carbon monoxide preferentially adsorbs onto platinum. This adsorption occurs via two bonds:
a σ bond between a p orbital of CO and a vacant d orbital of the metal;
π back-bonding between a full d orbital and an empty antibonding orbital of CO.

The strength of this latter bond depends on the capacity of the metal to donate electrons. Thus, in the case of a metallic particle which is enriched in electrons, back-donation is stronger and the C—O bond is weakened: the wave number of the C—O bond falls.

In the case of metallic particles, it is observed that the wave number of the C—O bond, ν_{CO}, varies with the degree of overlap. This phenomenon is explained by the perturbation caused by dipolar coupling between adsorbed molecules. To compensate for this perturbation, the wave number for the C—O bond is extrapolated to a zero degree of overlap. This value then provides information regarding the electron density of the particles.

In practice, an analysis of the displacement of the vibration band for the C—O bond in the zone corresponding to the adsorption of carbon monoxide onto metal particles, using the method described by Primet et al in Journal of Catalysis 88, (1984), pp 273-282, can be used to obtain the wave number for the C—O bond at zero degree of overlap.

### SUMMARY OF THE INVENTION

The invention concerns a catalyst comprising at least one metal M from the platinum group, tin, a phosphorus promoter, a halogenated compound, a porous support and at least one promoter X1 selected from the group constituted by gallium, indium, thallium, arsenic, antimony and bismuth. The catalyst has a ³¹P Magic angle spinning NMR signal in the range −30 to −50 ppm with respect to the signal for H₃PO₄. It also has a wave number for the carbon monoxide bond at zero degree of overlap of more than 2077 cm⁻¹. The invention also concerns the preparation of said catalyst and its use in catalytic reforming or aromatics production reactions.

### DETAILED DESCRIPTION OF THE INVENTION

The invention concerns a catalyst comprising at least one metal M from the platinum group, tin, a phosphorus promoter, a halogenated compound, a porous support and at least one promoter X1 selected from the group constituted by gallium, indium, thallium, arsenic and antimony, preferably from the group constituted by gallium, thallium and indium, highly preferably from the group constituted by gallium and indium, said catalyst having a ³¹P Magic angle spinning NMR signal in the range −30 to −50 ppm with respect to the signal for H₃PO₄.

The catalysts of the invention produce improved catalytic performances. In particular, the selectivity of said catalysts is increased towards the formation of C5+ compounds (i.e. compounds comprising at least 5 carbon atoms), while coke formation is substantially reduced.

The catalyst preparation process comprises a step for introducing phosphorus and the promoter or promoters X1 during a support preparation step. The signals observed in ³¹P MAS NMR which are characteristic of the catalysts of the invention are obtained if the phosphorus and the element or elements X1 are introduced together during synthesis or during shaping of the support.

The atomic ratio Sn/M is generally in the range 0.5 to 4.0, more preferably in the range 1.0 to 3.5, and highly preferably in the range 1.3 to 3.2. The ratio X1/M is generally in the range 0.1 to 5.0, more preferably in the range 0.2 to 3.0, and highly preferably in the range 0.4 to 2.2. The ratio P/M is generally in the range 0.2 to 30.0, more preferably in the range 0.5 to 20.0, and highly preferably in the range 1.0 to 15.0. The quantity of metal M is generally in the range 0.01% to 5% by weight, more preferably in the range 0.01% to 2% and still more preferably in the range 0.1% to 1% by weight.

The metal M is generally platinum or palladium, highly preferably platinum. The halogenated compound is generally selected from the group constituted by fluorine, chlorine, bromine and iodine. The quantity of halogenated compound is generally in the range 0.1% to 15.0% by weight, more preferably in the range 0.1% to 8.0% by weight, still more preferably in the range 0.2% to 5% by weight. If the halogenated compound is chlorine, the quantity of chlorine is generally in the range 0.0 to 5.0% by weight, preferably in the range 0.5% to 2.0% by weight.

The ³¹P MAS NMR and ¹H→³¹P CP MAS techniques were applied to our various samples. They were used to reveal in the first place the existence, for the catalysts having optimized catalytic performances, of a signal with a chemical shift in the range −30 to −50 ppm in ³¹P MAS NMR spectrum with respect to H₃PO₄ as the reference. Secondly, a combination of MAS and CP MAS analyses was also used for these catalysts to demonstrate a large gain in the ³¹P NMR signal with a chemical shift in the range 0 to −7 ppm. This signal corresponds to a portion of the surface phosphorus which is protonated and is characteristic of the manner in which the support is prepared.

The spectra were obtained using a Bruker DSX 400 MHz spectrometer using a 4 mm MAS probe. The samples were analyzed in the oxidized form. The spinning frequency was fixed at 10 to 12 kHz for the two types of experiment (³¹P MAS and ¹H→³¹P CP MAS) the ¹H→³¹P CP MAS spectra were obtained by swinging the magnetization on the proton by π/2 for a time in the range 2 to 5 μsec. The CP contact times used were optimized to satisfy Hartmann Hahn conditions. The chemical shifts were expressed with respect to those of H₃PO₄, used as the reference.

The infrared spectroscopy analyses were carried out on a Nexus 1 spectrometer. Prior to adsorption of CO, the samples were pre-treated by means of a temperature rise of 25° C. to 450° C. over 4 h with a constant temperature stage of 1 h at 150° C., then left at 450° C. under high vacuum for 10 h. They were then reduced at 450° C., for 30 min in excess H₂. Next, a high vacuum was applied for 15 min. The reduction procedure was carried out 4 times.

CO pulse adsorption was carried out at ambient temperature, then the carbon monoxide was desorbed at 25° C., 50° C., 75° C., 100° C. and 150° C. At each temperature, an infrared spectrum was recorded. Next, the method described by Primet et al in Journal of Catalysis 88, (1984), pp 273-282 was used to extrapolate the wave number for the C—O bond to a zero degree of overlap, ν⁰_{CO}.

These measurements showed that the optimized aromatics yields in the reaction for the catalytic reforming of naphtha are attributed to catalysts with a reduced electron density on the metal M, which gives rise to a ν⁰_{CO} at zero degree of overlap of strictly greater than 2077 cm⁻¹.

The support generally comprises at least one oxide selected from the group constituted by oxides of magnesium, titanium, zirconium, aluminium and silicon. Preferably, it is silica, alumina or silica-alumina, and highly preferably alumina. According to the invention, said porous support is advantageously in the form of beads, extrudates, pellets or powder. Highly advantageously, said support is in the form of beads or extrudates. The pore volume of the support is preferably in the range 0.1 to 1.5 cm³/g, more preferably in the range 0.4 to 0.8 cm³/g. Further, said porous support has a specific surface area which is advantageously in the range 50 to 600 m²/g, preferably in the range 100 to 400 m²/g, or even in the range 150 to 300 m²/g.

The invention also concerns a process for preparing the catalyst of the invention, comprising the following steps:
a) introducing the promoter or promoters X1 and phosphorus during one of sub-steps a1) or a2), said sub-step a1) corresponding to synthesis of a precursor of the main oxide, said sub-step a2) corresponding to shaping the support;
b) introducing tin during at least one of the sub-steps a1) and a2), the steps a) and b) possibly being consecutive or simultaneous;
c) drying the product obtained at the end of step b);
d) calcining the product obtained in step c) at a temperature in the range 350° C. to 650° C.;
e) depositing at least one metal M from the platinum group;
f) drying in a stream of neutral gas or a stream of gas containing oxygen, at a moderate temperature not exceeding 150° C.;
g) calcining the product obtained in step f) at a temperature in the range 350° C. to 650° C.

The tin may only be introduced in part when shaping the support, the process then comprising a supplemental step for depositing a complementary fraction of tin onto the support, either between steps d) and e), followed or otherwise by drying and calcining, or between steps e) and f), or after step g), followed by drying and calcining.

The calcining of step g) is generally carried out in the presence of air, optionally enriched with oxygen or nitrogen.

The promoters X1, P and Sn may be introduced using any technique which is known to the skilled person. During their introduction into the support, the promoters X1, P and Sn may be added by mixing, co-precipitating or dissolving; these methods are not limiting.

Thus, introduction of the tin may be simultaneous or may take place separately, before or after that for the precursors X1 and P.

In the case of introducing the promoter or promoters X1 and phosphorus, i.e. during synthesis of the oxide precursor, in accordance with a preferred method for preparation in accordance with the invention, the tin, phosphorus and the precursor or precursors X1 are introduced during synthesis of the precursor of the main oxide using a sol-gel type technique.

In accordance with another preferred method, the precursors are added to a prepared sol of a main oxide precursor.

The support is shaped using prior art support shaping techniques, such as shaping procedures involving extrusion or oil drop coagulation.

The X1 precursors are of a plurality of types depending on the nature of X1 and may be used alone or as a mixture. In the case of indium, indium halides, nitrates, sulphates, perchlorate, cyanide or hydroxide are suitable. Precursors of the gallium halide, nitrate, sulphate, cyanide, hydroxide and oxyhalide type may be used. Thallium may be introduced in the form of thallium nitrates, sulphates and hydroxide. In the case of antimony, antimony nitrates, sulphates and hydroxide are suitable. Precursors of arsenic halides and oxyhalides may be used. Bismuth may be introduced in the form of bismuth halides, nitrates, hydroxide, oxyhalides or carbonate, or as bismuthic acid.

The tin precursors may be minerals or may be organometallic in type, possibly of the hydrosoluble organometallic type. Various precursors may be used, alone or as a mixture. In particular, tin may be selected; in a non-limiting manner, the tin may be selected from the group formed by halogenated, hydroxide, carbonate, carboxylate, sulphate, tartrate and nitrate compounds. These forms of tin may be introduced into the catalyst preparation medium as they are or they may be generated in situ (for example by introducing tin and carboxylic acid). Examples of organometallic tin-based type precursors are SnR₄, where R represents an alkyl group, for example the butyl, Me₃SnCl, Me₂SnCl₂, Et₃SnCl, Et₂SnCl₂, EtSnCl₃, iPrSnCl₂ group, and the hydroxides Me₃SnOH, Me₂Sn(OH)₂, Et₃SnOH, Et₂Sn(OH)₂, the oxides (Bu₃Sn)₂O, the acetate Bu₃SnOC(O)Me. Preferably, halogenated species, in particular chlorinated species of tin, are used. In particular, SnCl₂ or SnCl₄ are advantageously used.

Having introduced the promoters Sn, X1 and P into the support or onto the support that has already been shaped in the case of tin, the protocol for preparing the catalysts of the invention necessitates calcining before depositing the metal M from the platinum group (step d). Said calcining is preferably carried out at a temperature in the range 350° C. to 650° C., preferably in the range 400° C. to 600° C. and more preferably in the range 400° C. to 550° C. The temperature rise may be regular, or may include intermediate constant temperature stages, said stages being reached with fixed or variable temperature profiles. These rises in temperature may thus be identical or differ in their rate (in degrees per minute or per hour). The gas atmosphere used during calcining contains oxygen, preferably in the range 2% to 50% by volume and more preferably in the range 5% to 25%. Air may thus also be used during this calcining step.

After obtaining the support, at least one metal M from the platinum group is deposited (step e). In this step, the metal M may be introduced by dry impregnation or excess solution impregnation, using a precursor or a mixture of precursors containing a metal M from the platinum group. Impregnation may be carried out in the presence of species acting on the interaction between the precursor of the metal M and the support. In a non-limiting manner, said species may be mineral acids (HCl, HNO₃) or organic acids (carboxylic or polycarboxylic acid types), and organic complexing type compounds. Preferably, impregnation is carried out using any technique which is known to the skilled person for obtaining a homogeneous distribution of the metal M within the catalyst.

The precursors of the metal M form part of the following group, although this list is not limiting: hexachloroplatinic acid, bromoplatinic acid, ammonium chloroplatinate, platinum chlorides, platinum dichlorocarbonyl dichloride, and platinum tetramine chloride.

At this stage, the catalyst containing X1, Sn, P and platinum is dried (step f), in a neutral atmosphere or an atmosphere containing oxygen (air may be used), at a moderate temperature which preferably does not exceed 250° C. Preferably, drying is carried out at a temperature of 200° C. or less and over a period of a few minutes to a few hours.

This step is then followed by calcining the product obtained in step f). Said calcining is preferably carried out in the presence of air. This air may also be enriched in oxygen or nitrogen. Preferably, the oxygen content in said gas reaches 0.5% to 30.0% by volume, more preferably in the range 2% to 25%.

Said calcining is carried out at a temperature in the range 350° C. to 650° C., preferably in the range 400° C. to 650° C., and more preferably in the range 450° C. to 550° C. The temperature profile may optionally contain constant temperature stages.

When the various precursors used in the preparation of the catalyst of the invention do not contain halogen or contain halogen in insufficient quantities, it may be necessary to add a halogenated compound during the preparation. Any compound which is known to the skilled person may be used and incorporated into any one of the steps for preparing the catalyst of the invention. In particular, it is possible to use compounds of the Friedel-Crafts type such as aluminium chloride or bromide. It is also possible to use organic compounds such as methyl or ethyl halides, for example dichloromethane, chloroform, dichloroethane, methyl chloroform or carbon tetrachloride.

The chlorine may also be added to the catalyst of the invention using an oxychlorination treatment. Said treatment may, for example, be carried out at 500° C. for 4 hours in a flow of air containing the quantity of gaseous chlorine necessary to deposit the desired quantity of chlorine and a quantity of water with a H₂O/Cl molar ratio close to 20, for example.

The chlorine may also be added by means of impregnation with an aqueous hydrochloric acid solution. A typical protocol consists of impregnating the solid so as to introduce the desired quantity of chlorine. The catalyst is maintained in contact with the aqueous solution for a period sufficiently long to deposit this quantity of chlorine, then the catalyst is drained and dried at a temperature in the range 80° C. to 150° C., then finally calcined in air at a temperature in the range 450° C. to 650° C.

The invention also concerns the use of a catalyst in a catalytic reforming reaction or an aromatics production reaction by bringing said catalyst into contact with a hydrocarbon feed. Reforming processes can be used to increase the octane number, of gasoline fractions deriving from the distillation of crude oil and/or from other refining processes such as catalytic cracking or thermal cracking, for example.

Processes for the production of aromatics produce base products (benzene, toluene, xylenes) which can be used in petrochemistry.

These two processes are of additional interest as they contribute to the production of large quantities of the hydrogen which is indispensable to the hydrogenation and hydrotreatment processes carried out at the refinery. These two types of process can be distinguished by the choice of operating conditions and the composition of the feed; these are familiar to the skilled person.

The feed for the reforming processes generally contains paraffinic, naphthenic and aromatic hydrocarbons containing 5 to 12 carbon atoms per molecule. Said feed is defined, inter alia, by its density and its composition by weight. These feeds may have an initial boiling point in the range 40° C. to 70° C. and an end point in the range 160° C. to 220° C. They may also be constituted by a fraction or mixture of gasoline fractions with initial boiling points and end points in the range 40° C. to 220° C. The feed may also be constituted by a heavy naphtha with a boiling point in the range 160° C. to 200° C.

Typically, the reforming catalyst is charged into a unit and undergoes a prior reduction treatment. This reduction step is generally carried out in a dilute or pure hydrogen atmosphere and at a temperature which is advantageously in the range 400° C. to 600° C., preferably in the range 450° C. to 550° C.

The feed is then introduced, in the presence of hydrogen, and with a hydrogen/feed hydrocarbons molar ratio which is generally in the range 0.1 to 10, preferably in the range 1 to 8.

The operating conditions for reforming are generally as follows: a temperature which is preferably in the range 400° C. to 600° C., more preferably in the range 450° C. to 540° C., and a pressure which is preferably in the range 0.1 MPa to 4 MPa, more preferably in the range 0.25 MPa to 3.0 MPa. All or a portion of the hydrogen produced may be recycled to the inlet to the reforming reactor.

### EXAMPLES

The following examples illustrate the invention.

### Example 1 (Comparative)

### Preparation of a Catalyst A: Pt/(Al₂O₃—Sn)—Cl

A support in the form of alumina beads containing 0.3% by weight of tin and with a mean diameter of 1.2 mm was prepared by bringing tin dichloride into contact with an alumina hydrosol obtained by hydrolysis of aluminium chloride. The alumina hydrosol obtained thereby was then passed into a vertical column filled with additive oil. The spheres thus obtained were heat treated at up to 600° C. in order to obtain beads with good mechanical strength. The support obtained thereby had a BET surface of 205 m²/g.

A catalyst A was prepared on this support by depositing 0.3% by weight of platinum and 1% by weight of chlorine onto the final catalyst. 400 cm³ of an aqueous solution of hexachloroplatinic acid and hydrochloric acid was added to 100 g of alumina support containing tin. It was left in contact for 4 hours then drained. It was dried at 120° C. then calcined for 2 hours at 500° C. in a flow of air of 100 litres per hour, with a temperature ramp-up of 7° C. per minute. The quantity of tin tetrachloride was selected so as to obtain a total of 0.3% by weight of tin on the calcined product. The catalyst A obtained after calcining contained 0.29% by weight of platinum, 0.30% by weight of tin and 1.02% by weight of chlorine.

### Example 2 (Comparative)

### Preparation of a Catalyst B: Pt/(Al₂O₃—Sn—In)—Cl

A support in the form of alumina beads containing 0.3% by weight of tin and 0.3% by weight of indium with a mean diameter of 1.2 mm was prepared by bringing tin dichloride and indium nitrate into contact with an alumina hydrosol obtained by hydrolysis of aluminium chloride. The alumina hydrosol obtained thereby was then passed into a vertical column filled with additive oil. The spheres thus obtained were heat treated at up to 600° C. in order to obtain beads with good mechanical strength. The support obtained thereby had a BET surface of 201 m²/g.

A catalyst B was prepared on this support, aiming for the same platinum and chlorine contents as in Example 1. The catalyst B obtained after calcining contained 0.29% by weight of platinum, 0.29% by weight of tin, 0.30% by weight of indium and 1.05% by weight of chlorine.

### Example 3 (Comparative)

### Preparation of a Catalyst C: Pt/(Al₂O₃—Sn—P)—Cl

A support in the form of alumina beads containing 0.3% by weight of tin and 0.4% by weight of phosphorus and with a mean diameter of 1.2 mm was obtained in a manner similar to that described in Example 1 by bringing tin dichloride and phosphoric acid into contact with an alumina hydrosol. The support obtained thereby had a BET surface of 198 m²/g.

A catalyst C was prepared on this support, aiming for the same platinum and chlorine contents as in Example 1. The catalyst C obtained after calcining contained 0.30% by weight of platinum, 0.31% by weight of tin, 0.39% by weight of phosphorus and 1.00% by weight of chlorine.

### Example 4 (in accordance with the invention)

### Preparation of a Catalyst D: Pt/(Al₂O₃—Sn—In—P)—Cl

A support in the form of alumina beads containing 0.3% by weight of tin, 0.3% by weight of indium and 0.4% by weight of phosphorus and with a mean diameter of 1.2 mm was obtained in a manner similar to that described in Example 1 by bringing tin dichloride, indium nitrate and phosphoric acid into contact with an alumina hydrosol The support obtained thereby had a BET surface of 196 m²/g.

A catalyst D was prepared on this support, aiming for the same platinum and chlorine contents as in Example 1. The catalyst D obtained after calcining contained 0.30% by weight of platinum, 0.31% by weight of tin, 0.32% by weight of indium, 0.38% by weight of phosphorus and 1.00% by weight of chlorine.

### Example 5 (in accordance with the invention)

### Preparation of a Catalyst E: Pt/(Al₂O₃—Sn—In—P)—Cl

A support in the form of alumina beads was prepared in the same manner as in Example 4, with the same quantities of tin and phosphorus, but only introducing 0.2% by weight of indium. The support obtained thereby had a BET surface of 210 m²/g.

A catalyst E was prepared on this support, aiming for the same platinum and chlorine contents as in Example 1. The catalyst E obtained after calcining contained 0.31% by weight of platinum, 0.31% by weight of tin, 0.22% by weight of indium, 0.40% by weight of phosphorus and 1.02% by weight of chlorine.

### Example 6 (Comparative)

### Preparation of a Catalyst F: Pt—In/(Al₂O₃—Sn—P)—Cl

A support was prepared, aiming for the same quantities of tin and phosphorus as in Example 3. The support obtained thereby had a BET surface of 180 m²/g.

A catalyst F was prepared on this support, aiming for 0.3% by weight of platinum, 0.3% by weight of indium and 1% by weight of chlorine on the final catalyst.

400 cm³ of an aqueous solution of hexachloroplatinic acid and hydrochloric acid was added to 100 g of alumina support containing tin and phosphorus. It was left in contact for 4 hours then drained. It was dried at 90° C. then brought into contact with 200 cm³ of an aqueous solution of indium nitrate in the presence of hydrochloric acid. It was left in contact for 4 hours, drained, dried at 120° C. then calcined for 2 hours at 500° C. in a flow of air of 100 litres per hour, with a temperature ramp-up of 7° C. per minute. The catalyst F obtained after calcining contained 0.30% by weight of platinum, 0.32% by weight of tin, 0.29% by weight of indium, 0.41% by weight of phosphorus and 1.04% by weight of chlorine.

### Example 7 (Comparative)

### Preparation of a Catalyst G: Pt—In—P/(Al₂O₃—Sn)—Cl

A support was prepared, aiming for the same quantities of tin as in Example 1.

A catalyst G was prepared on this support, aiming for 0.3% by weight of platinum, 0.3% by weight of indium, 0.4% by weight of phosphorus and 1% by weight of chlorine on the final catalyst. The support obtained thereby had a BET surface of 209 m²/g.

400 cm³ of an aqueous solution of hexachloroplatinic acid and hydrochloric acid was added to 100 g of alumina support containing tin and phosphorus. It was left in contact for 4 hours then drained. It was dried at 90° C. then brought into contact with 200 cm³ of an aqueous solution of indium nitrate and phosphoric acid in the presence of hydrochloric acid. It was left in contact for 4 hours, drained, dried at 120° C. then calcined for 2 hours at 500° C. in a flow of air of 100 litres per hour, with a temperature ramp-up of 7° C. per minute. The catalyst G obtained after calcining contained 0.30% by weight of platinum, 0.31% by weight of tin, 0.33% by weight of indium, 0.38% by weight of phosphorus and 1.05% by weight of chlorine.

### Example 8 (in accordance with the invention)

### Preparation of a Catalyst H: Pt—Sn/(Al₂O₃—Sn—In—P)—Cl

A support was prepared, aiming for the same quantities of indium and phosphorus as in Example 4, but with 0.2% by weight of tin. The support obtained thereby had a BET surface of 182 m²/g.

A catalyst H was prepared on this support by depositing 0.35% by weight of platinum, a supplemental 0.2% by weight of tin in order to obtain 0.4% by weight of tin and 1% by weight of chlorine on the final catalyst.

400 cm³ of an aqueous solution of hexachloroplatinic acid and hydrochloric acid was added to 100 g of alumina support containing tin and indium. It was left in contact for 4 hours then drained. It was dried at 90° C. then brought into contact with 200 cm³ of an aqueous solution of tin tetrachloride in the presence of hydrochloric acid. It was left in contact for 4 hours, drained, dried at 120° C. then calcined for 2 hours at 500° C. in a flow of air of 100 litres per hour, with a temperature ramp-up of 7° C. per minute. The catalyst H obtained after calcining contained 0.36% by weight of platinum, 0.41 by weight of tin, 0.29% by weight of indium, 0.41% by weight of phosphorus and 0.99% by weight of chlorine.

### Example 9 (n accordance with the invention)

### Preparation of a Catalyst I: Pt—Sn/(Al₂O₃—Sn—Sb—P)—Cl

An alumina bead support containing 0.1% by weight of tin, 0.4% by weight of antimony and 0.4% by weight of phosphorus and with a mean diameter of 1.2 mm was prepared in a manner similar to that described in Example 4 using tin dichloride, gallium nitrate and phosphoric acid. The support obtained thereby had a BET surface of 191 m²/g.

A catalyst I was prepared from said support, with the same quantities of platinum, tin and chlorine as in Example 7. Catalyst G obtained after calcining contained 0.29% by weight of platinum, 0.30% by weight of tin, 0.32% by weight of indium, 0.42% by weight of phosphorus and 1.10% by weight of chlorine.

### Example 10

### Infrared and NMR Characterizations of Catalysts A to I

The values for the ³¹P NMR signals of catalysts C to I, determined using the methods presented in the description, as well as the gains in area of the various signals in the {¹H-³¹P} CP MAS series are detailed in Table 1. The gains were calculated as the ratio between the area of the signal obtained in cross polarization (CP MAS) and that of the signal with the same chemical shift in direct polarization (MAS).

The ν⁰_{CO} values for the 9 catalysts are also reported in this table.

TABLE 1
Infrared and NMR characterizations of catalysts A to I

| | | ³¹P NMR characterization | |
|---|---|---|---|
| | IR characterization | ³¹P MAS | ¹H → ³¹P CP MAS |
| Catalyst | ν⁰_{CO} (cm⁻¹) | δ (ppm) | Gain in area of signal |
| A, comparative | 2071 | * | * |
| B, comparative | 2075 | * | * |
| C, comparative | 2073 | −3 | 1.0 |
| | | −9 | 2.2 |
| | | −21 | 0.8 |
| D, invention | 2089 | −4 | 8.3 |
| | | −11 | 1.0 |
| | | −19 | 1.1 |
| | | −40 | 1.0 |
| E, invention | 2085 | −3 | 4.2 |
| | | −11 | 1.0 |
| | | −19 | 0.9 |
| | | −40 | 1.0 |
| F, comparative | 2075 | −3 | 1.0 |
| | | −9 | 2.3 |
| | | −21 | 0.9 |
| G, comparative | 2071 | −3 | 1.0 |
| | | −9 | 3.5 |
| | | −19 | 0.9 |
| H, invention | 2087 | −4 | 8.1 |
| | | −11 | 1.0 |
| | | −20 | 1.0 |
| | | −40 | 0.9 |
| I, invention | 2082 | −3 | 7.9 |
| | | −11 | 1.0 |
| | | −19 | 1.1 |
| | | −38 | 1.0 |

* Catalysts A and B contain no P, and so phosphorus NMR was not carried out on them.

### Example 11

### Evaluation of Performances of Catalysts A to I in Catalytic Reforming

Samples of the catalysts prepared as described in Examples 1 to 9 were placed in a reaction bed adapted to the conversion of a hydrocarbon feed of the naphtha type derived from oil distillation. This naphtha had the following composition (by weight):
52.6% of paraffinic compounds;
31.6% of naphthenes;
15.8% of aromatic molecules;
   with a total density of 0.759 g/cm³.

The research octane number of the feed was close to 55.

After loading into the reactor, the catalysts were activated by heat treatment in an atmosphere of pure hydrogen for a period of 2 h at 490° C.

The catalytic performances were evaluated under reforming reaction conditions in the presence of hydrogen and the naphtha described above. In particular, the conditions for use and for comparison of the catalysts were as follows:
pressure of the reactor kept at 8 bar g (0.8 MPa g);
flow rate of feed of 2.0 kg/h per kg of catalyst;
hydrogen/hydrocarbon molar ratio of feed: 4.

The comparison was made at iso-quality of research octane number of the liquid effluents (also termed reformates) resulting from catalytic conversion of the feed. The comparison was carried out for a research octane number of 104.

TABLE 2
Catalyst performances

| | C5+ | C4− | | | |
|---|---|---|---|---|---|
| | yield at | yield at | Aromatics | | Coke |
| | 148 h | 148 h | yield at 148 h | Deactivation | (% by |
| Catalyst | (wt %) | (wt %) | (wt %) | (° C./h) | weight/h) |
| A | 88.38 | 8.37 | 76.26 | +0.088 | +0.034 |
| B | 88.79 | 8.05 | 76.52 | +0.140 | +0.038 |
| C | 88.29 | 8.39 | 76.40 | +0.099 | +0.033 |
| D | 89.36 | 7.34 | 76.91 | +0.084 | +0.026 |
| E | 89.12 | 7.58 | 76.90 | +0.099 | +0.030 |
| F | 88.64 | 8.11 | 76.49 | +0.102 | +0.034 |
| G | 88.51 | 8.23 | 76.25 | +0.092 | +0.038 |
| H | 89.22 | 7.45 | 76.88 | +0.085 | +0.029 |
| I | 89.25 | 7.48 | 76.77 | +0.089 | +0.029 |

FIG. 1 shows the change in yield of aromatics compounds as a function of displacement of the vibration frequency of the C—O bond, illustrating the gain in yield of aromatics products obtained when the electron density of the platinum particles is reduced under the conditions for recording the IR spectra.

A ν⁰_{CO} at zero degree of overlap strictly greater than 2077 cm⁻¹ allows improved aromatics yields to be obtained.

1. A process for preparing a catalyst comprising at least one metal M from the platinum group, tin, a phosphorus promoter, a halogenated compound, a porous support and at least one promoter X1 selected from the group constituted by gallium, indium, thallium, arsenic, antimony and bismuth, said process comprising the following steps:
a) introducing the promoter or promoters X1 and phosphorus during one of sub-steps a1) or a2), said sub-step a1) corresponding to synthesis of a precursor of the main oxide, said sub-step a2) corresponding to shaping the support;
b) introducing tin during at least one of the sub-steps a1) and a2), the steps a) and b) possibly being consecutive or simultaneous;
c) drying the product obtained at the end of step b);
d) calcining the product obtained in step c) at a temperature in the range 350° C. to 650° C.;
e) depositing at least one metal M from the platinum group;
f) drying in a stream of neutral gas or a stream of gas containing oxygen, at a moderate temperature not exceeding 150° C.;
g) calcining the product obtained in step f) at a temperature in the range 350° C. to 650° C.
h)

[symbol]

2. A process for preparing a catalyst according to claim 1, in which the atomic ratio Sn/M is in the range 0.5 to 4.0.

3. A process for preparing a catalyst according to claim 1, in which the ratio X1/M is in the range 0.1 to 5.0.

4. A process for preparing a catalyst according to claim 1, in which the ratio P/M is in the range 0.2 to 30.0.

5. A process for preparing a catalyst according to claim 1, in which the quantity of metal M is in the range 0.01% to 5% by weight.

6. A process for preparing a catalyst according to claim 1, in which the metal M is platinum or palladium.

7. A process for preparing a catalyst according to claim 1, in which the halogenated compound is selected from the group constituted by fluorine, chlorine, bromine and iodine.

8. A process for preparing a catalyst according to claim 1, in which the quantity of halogenated compound is in the range 0.1% to 15.0% by weight.

9. A process for preparing a catalyst according to claim 1, in which the halogenated compound is chlorine and the chlorine content is in the range 0.1% to 5.0% by weight.

10. A process for preparing a catalyst according to claim 1, in which the support comprises at least one oxide selected from the group constituted by oxides of magnesium, titanium, zirconium, aluminium and silicon.

11. A process for preparing a catalyst according to claim 1, in which the tin is only introduced in part during synthesis or shaping of the support, the process then comprising a supplemental step for depositing a complementary fraction of the tin onto the support, either between steps d) and e), followed or not followed by drying and calcining, or between steps e) and f), or after step g), followed by drying and calcining.

12. A process using a catalyst prepared in accordance with claim 1 in a reaction for catalytic reforming or aromatics production by bringing said catalyst into contact with a hydrocarbon feed.
